(19) United States
(12) Patent Application Publication
(10) Pub. No.: US 2018/0216193 A1
(43) Pub. Date: Aug. 2, 2018

(54) METHODS FOR PREDICTING THE SURVIVAL TIME AND TREATMENT RESPONSIVENESS OF A PATIENT SUFFERING FROM A SOLID CANCER

(71) Applicants: INSERM (INSTITUT NATIONAL DE LA SANTÉ ET DE LA RECHERCHE MÉDICALE), Paris (FR); UNIVERSITÉ DE TOURS FRANÇOIS RABELAIS, Tours (FR); CENTRE HOSPITALIER RÉGIONAL UNIVERSITAIRE DE TOURS, Tours (FR); NATIONAL AND KAPODISTRIAN UNIVERSITY OF ATHENS, Athens (FR)

(72) Inventors: Nathalie HEUZE VOURC'H, Tours Cedex 1 (FR); Emilie DALLONEAU, Tours Cedex 1 (FR); Konstantinos MAVRIDIS, Athens (GR); Valérie GOUILLEUX-GRUART, Tours (FR)

(21) Appl. No.: 15/746,550

(22) PCT Filed: Jul. 21, 2016
(86) PCT No.: PCT/EP2016/067424

§ 371 Date: Jan. 22, 2018

(30) Foreign Application Priority Data
Jul. 23, 2015 (EP) 15306197.3

(51) Int. Cl. C12Q 1/6886

(52) U.S. Cl. CPC C12Q 1/6886; C12Q 2600/106; C12Q 2600/118; C12Q 2600/158

(57) ABSTRACT

The present invention relates to a method for predicting the survival time of a patient suffering from a solid cancer comprising i) determining in a tissue sample obtained from the patient the gene expression level of FcRn ii) comparing expression level determined at step i) with their predetermined reference value and iii) providing a good prognosis when expression level determined at step i) is higher than their predetermined reference value, or providing a bad prognosis when expression levels determined at step i) are lower than their predetermined reference values.

### FIELD OF THE INVENTION

The present invention relates to methods and kits for predicting the survival time and responsiveness of a patient suffering from a solid cancer.

### BACKGROUND OF THE INVENTION

Cancer remains a serious public health problem in developed countries. Accordingly, to be most effective, cancer treatment requires not only early detection and treatment or removal of the malignancy, but a reliable assessment of the severity of the malignancy and a prediction of the likelihood of cancer recurrence. The stage of a cancer indicates how far a cancer has spread. Staging is important because treatment is often decided according to the stage of a cancer. To date, cancers are generally classified according to the UICC-TNM system. The TNM (for “Tumor-Node-Metastasis”) classification system uses the size of the tumor, the presence or absence of tumor in regional lymph nodes, and the presence or absence of distant metastases, to assign a stage to the tumor. The TNM system developed from the observation that patients with small tumours have better prognosis than those with tumours of greater size at the primary site. In general, patients with tumours confined to the primary site have better prognosis than those with regional lymph node involvement, which in turn is better than for those with distant spread of disease to other organs. Accordingly, cancer can be generally divided into four stages. Stage I is very localized cancer with no cancer in the lymph nodes. Stage II cancer has spread to the lymph nodes. Stage III cancer has spread near to where the cancer started. Stage IV cancer has spread to another part of the body. The assigned stage is used as a basis for selection of appropriate therapy and for prognostic purposes. For example chemotherapy is always recommended for patients with stage IV cancers. On the contrary, there are no relevant guidelines for prescribing chemotherapy for patient with a UICC-TNM stage II or III cancer. Accordingly there is a need for reliable diagnostic tools to guide treatment decisions is all the more as an essential step for the multitude of available new therapies is the efficient selection of patients for adequate cancer therapy.

The above TNM classifications, although they are to be useful, are imperfect and do not allow a reliable prognosis of the outcome of the cancers. Recently, Galon et al. suggested that analysing the expression of genes related to the adaptive immune response within the tumour may be suitable for predicting the outcome of a cancer in a patient (WO2007045996). Thus they provides list of genes and combination thereof that may be useful for the prognosis of patients for progression of cancer. However the methods depicted in said document fail to point out particular biomarker that provide a better performance than the TNM classification does for predicting the survival time of patient with a cancer and for predicting the treatment response of the patient, especially with an immunotherapeutic agent which will restore antitumor immune response.

Despite the intensive study of molecular mechanisms responsible for cancer development, there is still a lack of good prognostic or even better survival biomarkers and a need for more effective therapies (Liu et al. 2014). Recently, the development of targeted and personalized therapy has led researchers to the discovery of biomarkers representing a complementary strategy for predicting patients' outcomes and defined a specific treatment. But most of these biomarkers are based on tumor markers and give mostly indications to use a specific targeted therapy, but do not predict cancer overcome.

### SUMMARY OF THE INVENTION

The present invention relates to a method for predicting the survival time of a patient suffering from a solid cancer comprising i) determining in a tissue sample obtained from the patient the gene expression level of neonatal Fc receptor (FcRn) ii) comparing expression level determined at step i) with its predetermined reference value and iii)
providing a good prognosis when the expression level determined at step i) is higher than the predetermined reference value, or
providing a bad prognosis when the expression level determined at step i) are lower than the predetermined reference value.

The present invention also relates to a method for predicting the survival time of a patient suffering from a solid cancer comprising i) determining in a non-tumor sample or in a tumor sample obtained from the patient the gene expression level of neonatal Fc receptor (FcRn) ii) comparing every expression level determined at step i) with its predetermined reference value and iii)
providing a good prognosis when the expression level determined at step i) is higher than the predetermined reference value, or
providing a bad prognosis when the expression level determined at step i) are lower than the predetermined reference value.

The method is also suitable for predicting the responsiveness of the patient to a treatment.

### DETAILED DESCRIPTION OF THE INVENTION

The present inventors have assayed for a link between the expression of FcRn and the prognosis of solid tumor (NSCLC) using 80 patients with NSCLC disease.

As disclosed in the examples herein, the presence of FcRn protein was evaluated in a pooled protein extracts from 10 cancerous and 10 matched non-cancerous tissues by western blot. The distribution of the protein was studied by immunohistochemistry on lung tissues sections. ROC curve analyses were used to determine a cut-off value of FcRn mRNA expression unit to discriminate cancerous from non-cancerous tissues. Kaplan-Meier method and Cox regression were used to evaluate the relationship between FcRn mRNA expression, clinico-pathological features and overall survival.

More precisely, in NSCLC, FcRn was mainly found in resident and tumor infiltrating immune cells. The corresponding mRNA and protein are significantly less abundant in lung tumor than non-cancerous tissue. The ROC curve analysis revealed that choosing a cut-off value at 1.66 unit expression of FcRn mRNA allowed to effectively discriminate cancerous tissues from non-cancerous tissues. Moreover, a high expression of FcRn mRNA in cancerous and interestingly in non-cancerous tissues is an independent indicator of favorable overall survival for NSCLC patients.

These results support that FcRn mRNA and protein are down regulated in cancerous tissues from NSCLC patients compared to the adjacent non-cancerous tissues. This decreased expression of FcRn is associated with a poor prognosis for NSCLC patients. These findings indicate that the assessment of FcRn mRNA expression may be a useful additional marker for immunoscoring, to help in the decision-making process for NSCLC patients.

Method for Prognosis of a Patient Suffering from a Solid Cancer

Accordingly, the present invention relates to a method for predicting the survival time of a patient suffering from a solid cancer comprising i) determining in a tissue sample obtained from the patient the gene expression level of neonatal Fc receptor (FcRn) ii) comparing expression level determined at step i) with its predetermined reference value and iii)
providing a good prognosis when all expression levels determined at step i) is higher than their predetermined reference values, or
providing a bad prognosis when all expression levels determined at step i) is lower than their predetermined reference values or

In a particular embodiment, the invention also relates to a method for predicting the survival time of a patient suffering from a solid cancer comprising i) determining in a non-tumor sample or in a tumor sample obtained from the patient the gene expression level of neonatal Fc receptor (FcRn) ii) comparing expression level determined at step i) with its predetermined reference value and iii)
providing a good prognosis when the expression level determined at step i) is higher than the predetermined reference value, or
providing a bad prognosis when the expression level determined at step i) is lower than the predetermined reference value.

The inventors further stratified NSCLC patients, according to FcRn mRNA levels in both cancerous (C) and non-cancerous (NC) tissue. Overall survival periods were longer for high (h) than low (l) FcRn mRNA levels in both cancerous and non-cancerous tissues. Strikingly, none of the “double high” (C^{h}/NC^{h}) patients died during the follow-up period, whereas the survival probabilities progressively worsened over time for patients with one or both tissue types scored as FcRn-low (FIG. 2C). The findings were similar when the patients were grouped into those with high FcRn expression in at least one tissue type (Ch and/or NCh) and those with low FcRn expression in both tissue types (Cl/NCl): the latter group showed significantly worse outcomes (P=0.002) (FIG. 2D). Thus, analysis of FcRn expression in both tissues types can provide significant and robust prognostic information (HR=0.273, 95% CI=0.129-0.577, P=0.001 Table 1) about NSCLC patients, which is independent of the currently used conventional indicators and important clinicopathological variables.

Accordingly in a particular embodiment, the present invention also relates to a method for predicting the survival time of a patient suffering from a solid cancer comprising i) determining in a non-tumor sample and in a tumor sample obtained from the patient the gene expression level of neonatal Fc receptor (FcRn) ii) comparing every expression level determined at step i) with its predetermined reference value and iii)
providing a good prognosis when both expression levels determined at step i) in a non-tumor sample and in a tumor sample are higher than their predetermined reference values, or
providing a bad prognosis when both expression levels determined at step i) in a non-tumor sample and in a tumor sample are lower than their predetermined reference values or
providing an intermediate prognosis when at least one expression level determined at step i) in a non-tumor sample or in a tumor sample is higher than its predetermined value

The patient may suffer from any solid cancer. Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adenocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). In a particular embodiment, the cancer is lung cancer (e.g. small cell lung cancer, non-small cell lung cancer), breast cancer, uterine cancer ovarian cancer and melanoma. In a more particular embodiment, the lung cancer is non-small cell lung cancer.

In a particular embodiment colorectal cancer is exclude from the method of the invention.

The term “tissue sample” means “tumor sample” and/or “non tumor sample”.

The term “non tumor sample” means any tissue sample derived from the patient not located in the tumor of the patient. The non-tumor sample is typically taken from sites at least 3 cm away from the edge of the tumor in the tissue bearing the tumor.

The term “tumor sample” means any tissue tumor sample derived from the patient. Said tissue sample is obtained for the purpose of the in vitro evaluation. The sample can be fresh, frozen, fixed (e.g., formalin fixed), or embedded (e.g., paraffin embedded). In a particular embodiment the tumor sample may result from the tumor resected from the patient. In another embodiment, the tumor sample may result from a biopsy performed in the primary tumour of the patient or performed in metastatic sample distant from the primary tumor of the patient. For example an endoscopical biopsy performed in the lung of the patient affected by a lung cancer. Tumor samples encompass tumors cells but also immune cells, located in the immune islets, in the stromal and peri-vascular compartments.

The term “FcRn” known as “neonatal Fc receptor” or “IgG receptor FcRn large subunit p51” or “FcRn alpha chain” or “IgG Fc fragment receptor transporter alpha chain” or “major histocompatibility complex class I-like Fc receptor” or “neonatal Fc-receptor for Ig” means neonatal Fc receptor protein (NM_001136019→NP_001129491 for variant 1 and NM_004107→NP_004098 for variant 2, variants 1 and 2 encode the same protein, transcript variant X1 XM_005258656→XP_005258713 and X3 XM_005258657→XP_005258714 encoding different predicted proteins) which is encoded by FCGRT gene and belongs to the family of receptors for the Fc portion of IgG. FcRn is associated with the beta-2-microglobulin (P2m) and shares structural features with MHC class I molecules¹. The heterodimer binds IgG and albumin, at acidic pH (pH<6.5). The whole sequence of human FcRn gene (gene FCGRT) is referenced as Gene ID: 2217.

FcRn is expressed in many cells and tissues throughout life. It is found in endothelial and epithelial cells from various organs (including placenta, lung, intestine and brain) where it participates in the recycling and transcytosis of IgG^{2, 3}. This contributes to the long half-life of IgGs in biological fluids and their distribution in the human body^{2, 4-6} FcRn is also involved in the humoral immune response: present in the epithelia of mucosa, FcRn is important for the host immune response against both bacteria and virus⁷⁻¹⁰. FcRn allows virus-specific IgG to interact with pathogens in epithelial cell endosomes where it neutralizes virus¹⁰. FcRn in immune cells, including dendritic cells, macrophages, monocytes 11 and neutrophils¹², is involved in phagocytosis, antigen presentation and cross-presentation¹²⁻¹⁴.

The present invention thus includes determining the expression level of neonatal Fc receptor (FcRn) (EL_{FcRn}) and comparing said level with the predetermined reference level for FcRn (ELR_{FcRn}).

Measuring the expression level of a gene can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particularfluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dichlorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as ³H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

It will be appreciated by those of skill in the art that by appropriately selecting labelledprobe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®,e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from tumor and/or non-tumor cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TBP, HPRT1 and TFRC. TATA-binding protein (TBP) and hypoxanthine phosphoribosyltransferase 1 (HPRT1) were used as reference genesin the present study. This normalization allows the comparison of the expression level in one sample, e.g., a patient sample, to another sample, or between samples from different sources.

Predetermined reference values used for comparison may comprise “cut-off” or “threshold” values that may be determined as described herein. Each reference (“cut-off”) value for gene of interest may be predetermined by carrying out a method comprising the steps of

a) providing a collection of tumor tissue samples from patients suffering of cancer;

b) determining the expression level of the gene for each tumor tissue sample contained in the collection provided at step a);

c) ranking the tumor tissue samples according to said expression level

d) classifying said tumor tissue samples in pairs of subsets of increasing, respectively decreasing, number of members ranked according to their expression level,

e) providing, for each tumour tissue sample provided at step a), information relating to the actual clinical outcome for the corresponding cancer patient (i.e. the duration of the disease-free survival (DFS) or the overall survival (OS) or both);

f) for each pair of subsets of tumour tissue samples, obtaining a Kaplan Meier percentage of survival curve;

g) for each pair of subsets of tumour tissue samples calculating the statistical significance (p value) between both subsets

h) selecting as reference value for the expression level, the value of expression level for which the p value is the smallest.

i) optionally determining the predictive/discriminatory capacity of the above mentioned cut-off value using an external cohort comprising of similar samples and patient characteristics or using publicly available datasets from microarray/next generation sequencing experiments (e.g. KM plotter, SurvExpress database, CAARRAY database) for cancer patients in general or for specific cancer subgroups (e.g. Stage I patients, patients with no adjuvant treatment, or patients with a specific histotype).

The reference value is selected such as the discrimination based on the criterion of the minimum p value is the strongest. In other terms, the expression level corresponding to the boundary between both subsets for which the p value is minimum is considered as the reference value. For categorization of FcRn expression levels, the generation of an optimal cut-off point, as described above is needed. It should be noted that the reference value is not necessarily the median value of expression levels.

In routine work, the reference value (cut-off value) may be used in the present method to discriminate between tumour/non tumor samples and therefore low/high risk patients.

Kaplan-Meier curves of percentage of survival as a function of time are commonly to measure the fraction of patients living for a certain amount of time after treatment and are well known by the man skilled in the art.

The man skilled in the art also understands that the same technique of assessment of the expression level of a gene should of course be used for obtaining the reference value and thereafter for assessment of the expression level of a gene of a patient subjected to the method of the invention.

For instance in the NSCLC and with the expression level of FcRn gene assessed by qPCR, the ROC curve analysis revealed that choosing a cut-off value at 1.66 unit expression of FcRn mRNA allowed to effectively discriminate cancerous tissues from non-cancerous tissues and which could be used as predetermined reference level for FcRn.

For instance in the NSCLC and with the expression level of FcRn gene assessed by array method, the validity of using a pre-determined cut-off value was largely validated using data from publicly available databases (FIG. 3). More precisely, a total of 1926 patients from the KM plotter database could be effectively stratified to low- and high-risk groups based on a predetermined cut-off value for FcRn mRNA expression. Results were similar from other commonly used NSCLC databases such as PrognoScan, PROGgeneV2 and SurvExpress. Collectively, we were to identify a pooled HR from n=31 studies/databases of 0.70 (95% CI=0.65-0.74), P<0.0001, with lack of any statistical heterogeneity (Cochran Q=33.08, P=0.3191, I²=9.3%, 95% CI=0-42%), or any significant bias (Egger: bias=−0.04965, P=0.8918), thus reaching a safe conclusion that FcRn expression is a robust discriminator of the post-operative course of NSCLC patients regarding overall survival and can be applied with the same prognostic power to different patient cohorts.

Similarly, the cut-off value that was identified as optimal in the array method for stratifying high and low risk patients could be used as a robust prognostic indicator. As shown by the meta-analysis used in the example, this FcRn-based stratification method that included:

i) analysis based on the same cut-off as defined in this study where this was applicable/allowed in the database (e.g. KM plotter),

ii) using the “standard” median cut-off value for categorization, or even iii) analysis of FcRn as a continuous variable independently of cut-off value stratification, the FcRn measurements can be effectively applied to external datasets in order to extract useful prognostic information and thus could be practically used in any NSCLC patient cohort.

Method for Determining Whether a Patient Suffering from a Solid Cancer Will Respond to a Treatment

As widely described in the literature, FcRn has multifaceted roles. Binding IgGs, it participates in their recycling and transcytosis, thereby contributing to their long half-life in biological fluids and their distribution in the human body (2, 3, 6). Accordingly, it is critically involved in therapeutic monoclonal antibody pharmacokinetics (27-29), which is an important parameter in their therapeutic response (30, 31). FcRn displays also important functions in regulating immune responses. Present in the epithelia of mucosa, FcRn is important for the host immune response against both bacteria and virus enabling bidirectional transcytosis of IgG (8, 9). Moreover, it elicits MHCII presentation and MHCI cross-presentation of IgG-complexed antigen (32, 33). Altogether, those studies show that FcRn exerts major roles in conferring active humoral immunity and immune surveillance at mucosal sites and exploiting these functions of FcRn were already successfully used for the development of novel mucosa vaccination strategies (34, 35).

Accordingly, the method of the present invention may be suitable to discriminate patients between 2 groups: a first group of patients as “bad responders” (i.e. the treatment will have a limited (or moderate) impact on their survival)—for example with low FcRn expression in both tissue types (C^{l}/NC^{l}) and a second group as “good responders” (i.e. the treatment will have a significant impact on their survival)—for example the “double high” FcRn (Ch/NCh).

Accordingly a further aspect of the invention relates to a method for determining whether a patient suffering from a solid cancer will respond to a treatment comprising i) determining in a tumor sample obtained from the patient the gene expression level of FcRn ii) comparing expression level determined at step i) with their predetermined reference value and iii) concluding that the patient will significantly respond to the treatment when expression level determined at step i) is higher than their predetermined reference values, or concluding that the patient will not significantly respond to the treatment when expression level determined at step i) is lower than their predetermined reference values.

Intermediate conclusions may also be provided when at least one gene is higher than its corresponding predetermined reference value in a non-tumor sample and in a tumor sample. Every time that the expression level of a gene is higher than its predetermined reference value, better will be the response of the patient to the treatment.

The method as above described is particularly suitable for early advanced cancer patients (stage I and stage II according to the TNM classification) for whom there are not established guidelines for the treatment. The method as above described will full fill the need by providing a reliant tool for determining whether a patient with a non-metastatic patient could benefit of a treatment.

The treatment may consist of radiotherapy, chemotherapy or immunotherapy. The treatment may consist of an adjuvant therapy (i.e. treatment after chirurgical resection of the primary tumor) of a neoadjuvant therapy (i.e. treatment before chirurgical resection of the primary tumor).

The term “chemotherapeutic agent” refers to all chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

The term “immunotherapeutic agent,” as used herein, refers to a compound, composition or treatment that indirectly or directly enhances, stimulates or augments the body's immune response against cancer cells and/or that lessens the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the patient with an amount of immune cells (T cells, NK, cells, dendritic cells, B cells . . . ).

Immunotherapeutic agents can be non-specific, i.e. boost the immune system generally so that it becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly augment the immune system. Non-specific immunotherapeutic agents have been used alone as the main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-a), IFN-beta (IFN-beta) and IFN-gamma (IFN-y). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer.

Interleukins have also shown good activity in combination with IFN-α in the treatment of various cancers (Negrier et al., Ann Oncol. 2002 13(9):1460-8; Tourani et al, J Clin Oncol. 2003 21(21):398794).

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in patients undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), bacillus Calmette-Guerin (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumours. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies.

Monoclonal antibodies can be used alone or in combination with other immunotherapeutic agents or chemotherapeutic agents.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

The immunotherapeutic treatment may consist of an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the patient's circulating lymphocytes, or tumor infiltrated lymphocytes, are isolated in vitro, activated by lymphokines such as IL-2 or transuded with genes for tumor necrosis, and readministered (Rosenberg et al., 1988; 1989). The activated lymphocytes are most preferably be the patient's own cells that were earlier isolated from a blood or tumor sample and activated (or “expanded”) in vitro. This form of immunotherapy has produced several cases of regression of melanoma and renal carcinoma.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy.

Such methods can optionally further comprise the administration of one or more additional cancer therapies, such as, but not limited to, chemotherapies, and/or another radiotherapy and/or another immunotherapy.

### Combination with Other Prognosis Markers in Cancer

It will be appreciated by one skilled in the art that prognosis of cancer may be performed solely on the basis of the results obtained by a method provided herein. Alternatively, a physician may also consider other markers associated with the Immunological status of the cancer used in existing methods to prognoses cancer. Thus, results obtained using methods of the present invention may be compared to and/or combined with results from other tests, assays or procedures performed for the prognosis of cancer. Such comparison and/or combination may help provide a more refine prognosis.

For example, this novel marker, being associated with the immune cell in cancer tissue (even at an early stage) it could be combined with the recently described Immunoscore®²⁵, a method measuring the beneficial impact of the immune infiltrate on tumor outcome, which has emerged in colorectal cancer and may be relevant in other malignancies, such as lung cancer.

Accordingly the prognosis methods for the solid cancer of the present invention may be used in combination with the expression of genes related to the adaptive immune response within the tumour may be suitable for predicting the outcome of a cancer in a patient (WO2007045996). Said additional biological markers are selected from the group consisting of the following biological markers:

(i) Various Biological Markers

ICAM-2/CD102, 4-1BB/TNFRSF9, IFN-gamma R1, IFN-gamma R2, B7-1/CD80, IL-1 RI, IL-2 R alpha, BLAME/SLAMF8, IL-2 R beta, CCR3, CCR4, CCR5, CCR6, CCR7, CCR8, IL-7 R alpha, CCR9, CXCR1/IL-8 RA, CD2, CD3epsilon, CD3zeta, CD3gamma, CD4, CD4+/45RA−, IL-12 R beta 1, CD4+/45RO−, IL-12 R beta 2, CD4+/CD62L-/CD44, CD4+/CD62L+/CD44IL-17, CD5, Integrin alpha 4/CD49d, CD6, Integrin alpha E/CD103, CD8, Integrin alpha M/CD11b, CD8+/45RA−, Integrin alpha X/CD11c, CD8+/45RO−, Integrin beta 2/CD18, CD27/TNFRSF7, LAG-3, CD28, LAIR1, CD30/TNFRSF8, LAIR2, CD31/PECAM-1, CD40 Ligand/TNFSF5, NCAM-L1, CD43, NTB-A/SLAMF6, CD45, CD83, CD84/SLAMF5, RANK/TNFRSF11A, L-Selectin, CD229/SLAMF3, SIRP beta 1, CD69, SLAM, Common gamma Chain/IL-2 R gamma, CRACC/SLAMF7, CX3CR1, CXCR3, CXCR4, CXCR6, TNF RI/TNFRSF1A, TNF RII/TNFRSF1B, Fas/TNFRSF6, Fas Ligand/TNFSF6, TSLP, TSLP R, ICAM-1/CD54, IL-2, IFN-gamma, IL-4, IL-5, IL-10, IL-13,

(ii) Biological markers of Th1/Th2 cells:

Il-2R Common beta Chain, Common gamma Chain/IL-2 R gamma, IFN-gamma, IFN-gamma R1, IL-12, IFN-gamma R2, IL-12 R beta 1, IL-2, IL-12 R beta 2, IL-2 R alpha, IL-2 R beta, IL-24, TNF RI/TNFRSF1A, TNF RII/TNFRSF1B, IL-4 R, TNF-beta/TNFSF1B,

(iii) Biological Markers of the Interferon Family:

IFN alpha, IFN beta, IFN-alpha/beta R1, IFN-alpha/beta R2, IFN-gamma R1, IFN-gamma R2, IFN-alpha A, IFN-alpha/beta R2, IFN-alpha B2, IFN-beta, IFN-alpha C, IFN-gamma, IFN-alpha D, IFN-alpha G, IFN-omega, IFN-alpha H2,

(iv) Biological Markers of the Common Gamma Chain Receptor Family:

Common gamma Chain/IL-2 R gamma, IL-7 R alpha, IL-2, IL-9, IL-2 R alpha, IL-9 R, IL-2 R beta, IL-15, IL-15 R alpha, IL-21, IL-7, IL-21 R, IL-31,

(v) Biological Markers of the CX3C Chemokines & Receptors:

CX3C Chemokine Ligands, CX3CL1/Fractalkine,

CX3C Chemokine receptors, CX3CR1,

(vi) Biological Markers of CXC Chemokines & Receptors,

CXC Chemokine Ligands, CXCL13/BLC/BCA-1, CXCL11/I-TAC, CXCL14/BRAK, CXCL8/IL-8, CINC-1, CXCL10/IP-10/CRG-2, CINC-2, CINC-3, CXCL16, CXCL15/Lungkine, CXCL5/ENA, CXCL9/MIG, CXCL6/GCP-2, CXCL7/NAP-2, GRO, CXCL4/PF4, CXCL1/GRO alpha, CXCL12/SDF-1, CXCL2/GRO beta, Thymus Chemokine-1, CXCL3/GRO gamma,

CXC Chemokine Receptors, CXCR6, CXCR3, CXCR1/IL-8 RA, CXCR4, CXCR2/IL-8 RB, CXCR5,

(vii) Biological Markers of CC Chemokines & Receptors,

CC Chemokine Ligands, CCL21/6Ckine, CCL12/MCP-5, CCL6/C10, CCL22/MDC, CCL28, CCL3L1/MIP-1 alpha Isoform LD78 beta, CCL27/CTACK, CCL3/MIP-1 alpha, CCL24/Eotaxin-2, CCL4/MIP-1 beta, CCL26/Eotaxin-3, CCL15/MIP-1 delta, CCL11/Eotaxin, CCL9/10/MIP-1 gamma, CCL14a/HCC-1, MIP-2, CCL14b/HCC-3, CCL19/MIP-3 beta, CCL16/HCC-4, CCL20/MIP-3 alpha, CCL1/I-309/TCA-3, CCL23/MPIF-1, MCK-2, CCL18/PARC, CCL2/MCP-1, CCL5/RANTES, CCL8/MCP-2, CCL17/TARC, CCL7/MCP-3/MARC, CCL25/TECK, CCL13/MCP-4CC

Chemokine Receptors, CCR1, CCR7, CCR2, CCR8, CCR3, CCR9, CCR4, D6, CCR5, HCR/CRAM-A/B, CCR6

(viii) Biological Markers of CC Chemokine Inhibitors

CCI, CC Viral Chemokine Homologs, MCV-type II, MIP-II, MIP-I, MIP-III

(ix) Biological Markers of C Chemokines & Receptors

The C (gamma) subfamily lacks the first and third cysteine residues. Lymphotactin (also known as SCM-1 alpha) and SCM-1 beta are currently the only two family members. Both have chemotactic activity for lymphocytes and NK cells.

C Chemokine Ligands, XCL1/Lymphotactin

C Chemokine Receptors, XCR1

(x) Biological Markers of Other Interleukins

IL-12, IL-12 R beta 1, IL-12 R beta 2, IL-27, IL-15, IL-31 The additional biological marker are selected from the group consisting of: ACE, ACTB, AGTR1, AGTR2, APC, APOA1, ARF1, AXIN1, BAX, BCL2, BCL2L1, CXCR5, BMP2, BRCA1, BTLA, C3, CASP3, CASP9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL5, CCL7, CCL8, CCNB1, CCND1, CCNE1, CCR1, CCR10, CCR2, CCR3, CCR4, CCR5, CCR6, CCR7, CCR8, CCR9, CCRL2, CD154, CD19, CD1a, CD2, CD226, CD244, PDCD1LG1, CD28, CD34, CD36, CD38, CD3E, CD3G, CD3Z, CD4, CD40LG, CD5, CD54, CD6, CD68, CD69, CLIP, CD80, CD83, SLAMF5, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNNB1, CTSC, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR3, CXCR4, CXCR6, CYP1A2, CYP7A1, DCC, DCN, DEFA6, DICER1, DKK1, Dok-1, Dok-2, DOK6, DVL1, E2F4, EBI3, ECE1, ECGF1, EDN1, EGF, EGFR, EIF4E, CD105, ENPEP, ERBB2, EREG, FCGR3A, CGR3B, FN1, FOXP3, FYN, FZD1, GAPD, GLI2, GNLY, GOLPH4, GRB2, GSK3B, GSTP1, GUSB, GZMA, GZMB, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, ID1, ifna1, ifna17, ifna2, ifna5, ifna6, ifna8, IFNAR1, IFNAR2, IFNG, IFNGR1, IFNGR2, IGF1, IHH, IKBKB, IL10, IL12A, IL12B, IL12RB1, IL12RB2, IL13, IL13RA2, IL15, IL15RA, IL17, IL17R, IL17RB, IL18, IL1A, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin, alpha E (antigen CD103, human mucosal lymphocyte, antigen 1; alpha polypeptide), Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7, MAF, MAP2K1, MDM2, MICA, MICB, MKI67, MMP12, MMP9, MTA1, MTSS1, MYC, MYD88, MYH6, NCAM1, NFATC1, NKG7, NLK, NOS2A, P2X7, PDCD1, PECAM-, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO, RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STAT6, STK36, TAP1, TAP2, TBX21, TCF7, TERT, TFRC, TGFA, TGFB1, TGFBR1, TGFBR2, TIMP3, TLR1, TLR10, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2

The most preferred additional biological markers are selected from the group consisting of CD3, CD8, PDCD1.

### Kit for Performing the Method of the Invention

A further object of the invention relates to kits for performing the methods of the invention, wherein said kits comprise means for measuring the expression level of neonatal Fc receptor (FcRn) gene of the invention in the sample obtained from the patient.

Accordingly, the present invention also relates to a kit of the invention comprising means for determining the expression level of neonatal Fc receptor (FcRn).

In one embodiment, the present invention relates to a kit for predicting the survival time or a method for determining the response to a treatment, comprising:
at least a means for determining the expression level of neonatal Fc receptor (FcRn) and
instructions for use.

In a particular embodiment, the kit comprising:
amplification primers and/or probe for determining the expression level of neonatal Fc receptor (FcRn),
instructions for use.

The kits may include probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

Example of sequences of primers for determining the expression level of FcRn (use in the present invention) is

Forward primer:
(SEQ ID No 1)
5′-CCCTGGCTTTTCCGTGCTT-3′
Reverse primer:
(SEQ ID No 2)
5′-TGACGATTCCCACCACGAG-3

The invention will be further illustrated by the following figures and examples. However, these examples and figures should not be interpreted in any way as limiting the scope of the present invention.

### FIGURES

FIG. 1 (A) Ratios of FcRn mRNA in cancerous (C)/non-cancerous (NC) in paired tissues from the NSCLC patients (P=6.27×10⁻¹³, Wilcoxon Signed Ranks test). (B) Distribution of FcRn mRNA levels in non-cancerous and cancerous tissues (n=80; P=2.54×10⁻²¹, Mann-Whitney test). (C) FcRn protein revealed by western blotting in a pool of 10 patients with matched cancerous and adjacent non-cancerous tissue. Recombinant human FcRn protein was loaded as a positive control. Signals were quantified with ImageJ and normalized to that for α-tubulin. (D) ROC curve analysis for FcRn mRNA level in cancerous and non-cancerous lung tissue samples. AUC=0.934, SE=0.024, 95% CI=0.884-0.967, P<0.0001, calculations according to DeLong et al., 1988. Youden index J=0.863 (95% CI=0.775-0.925, BCa bootstrap interval, 1000 iterations). (E) ROC curve analysis for FcRn mRNA levels in stage I cancerous and in non-cancerous tissues (AUC=0.947, 95% CI=0.905-0.989, P<0.001, calculations according to DeLong et al., 1988. Youden index J=0.871 (95% CI=0.771-0.938, BCa bootstrap interval, 1000 iterations).

FIG. 2 (A) Kaplan-Meier overall survival analyses for FcRn mRNA level in cancerous tissue from NSCLC patients. Cut-off value=0.888 (74^{th} percentile of FcRn mRNA abundance in cancerous samples). (B) Kaplan-Meier overall survival analysis for FcRn mRNA level in non-cancerous tissue from NSCLC patients. Cut-off value=2.82 (43^{th} percentile of FcRn expression in normal tissues). (C-D) Kaplan-Meier overall survival analysis for NSCLC patients stratified according to FcRn mRNA level in cancerous and non-cancerous. (C) C^{h}/NC^{h}: Patients with high FcRn mRNA levels in both tissue parts. C^{h}/NC^{l} or C^{l}/NC^{h}: Patients with high FcRn mRNA expression only in the cancerous or the non-cancerous tissue, respectively. C^{l}/NC^{l}: Patients with low FcRn mRNA levels in both tissue parts. (D) C^{h} and/or NC^{h}: Patients with high FcRn mRNA levels in at least one tissue part (cancer and/or non-cancerous tissues). C^{l}/NC^{l}: Patients with low FcRn mRNA levels in both tissue parts. Cut-off values as described above.

FIG. 3 (A) Kaplan-Meier overall survival curves based on FcRn expression (high vs low), as assessed by the KM plotter expression analysis data. (B) Bias assessment plot for trials considered in the meta-analysis.

FIG. 4 Response to monoclonal antibody is altered in the absence of FcRn expression. B16F10-Luc melanoma cells (100 000 cells/animal) were injected in the tail vein of C57 BL/6 mice. At day 1, day 4 and day 8 after tumor induction animals were treated by i.v. injection with a therapeutic monoclonal antibody (200 μg, anti-tyrosinase-related protein-1 named TA99 from BioXcell) or an irrelevant control isotype (200 μg, IgG2a). Animals were sacrificed at day 18, lung excised and tumor lesions counted. Results are expressed as the Mean±SEM of tumor lesions counted in the lungs and analyzed using a Mann Withney non parametric test.

### EXAMPLE 1

Material & Methods

Lung Tissue Specimens

Specimens of lung tissue were collected from patients (all gave their informed consent) who underwent surgery for primary lung cancer at the Trousseau Hospital, Tours, France, between 2006 and 2011 (n° DC-2008-308). Samples of cancerous and non-cancerous tissues (80 patients) were selected by a pathologist. The non-cancerous tissue samples were taken from sites at least 3 cm away from the edge of the tumor. Histological diagnosis was performed, and tumors were graded according to the World Health Organization classification of lung tumors. Patient data were recorded in a database for statistical analysis. This study was conducted in accordance with the ethical standards of the Helsinki Declaration and French bioethical authorities.

Quantitative Real-Time PCR

Following RNA extraction (QIAsymphony RNA kit, Qiagen) and cDNA synthesis (High Capacity cDNA Reverse Transcription kit, Applied Biosystems), quantitative-PCR was carried out on the LightCycler 480 (Roche Diagnostics) using the 1×SYBR Premix Ex Taq (Takara Bio Inc) as described by Gueugnon et al¹⁷. TATA-binding protein (TBP) and hypoxanthine phosphoribosyltransferase 1 (HPRT1) were used as reference genes.

Western Blot

Protein extracts (40 μg) of a pool of 10 cancerous tissues and 10 matched non-cancerous tissues from NSCLC patients, chosen blindly, and human recombinant FcRn protein, were separated on a NUPAGE 4-12% Bis-Tris gel (Life Technologies) and then transferred onto PVDF (polyvinyldenedifluoride) membranes by electroblotting. Membranes were blocked and then incubated with anti-FcRn (Novus Biologicals) or anti-alpha tubulin as a loading control antibody (Abcam) under the conditions recommended by the manufacturers. After incubation with the appropriate conjugated-HRP secondary antibody, membranes were developed using an enhanced chemiluminescence western blotting detection reagent (Amersham Biosciences).

Immunohistochemical Analysis

Briefly, 10 μm-thick tissue sections were deparaffinized, rehydrated and subjected to heat antigen retrieval in a citrate buffer (pH 6.0). Samples were blocked for endogenous peroxidase activity in 3% hydrogen peroxide-methanol. The VECTASTAIN Elite ABC Kit (Goat IgG, VECTOR Laboratories) was used for immunostaining following the manufacturer's instructions. Tissue sections were incubated with anti-FcRn polyclonal antibody (diluted 1:400) (Novus Biologicals) overnight at 4° C. A standard avidin-biotinimmunoperoxidase method and diaminobenzine as chromogen (DakoCytomation) were used for visualization. Rabbit IgG, whole molecule (Jackson ImmunoResearch), was used as a negative control.

Statistical Analyses

Differences between FcRn expression values between groups of cancerous samples were assessed using the non-parametric Mann-Whitney U and Jonckheere-Terpstra tests. FcRn mRNA levels in paired NSCLC samples were compared with the non-parametric Wilcoxon signed-rank test. The DeLong et al. 18 method was used for ROC curve analyses. For survival analyses, optimal cut-off points were established using the X-tile algorithm.

Kaplan-Meier survival curves were analyzed for the overall survival of the 71 NSCLC patients with available follow-up data; the log-rank test was used for evaluation of statistical significance. Cox regression models were used to evaluate the prognostic potential of FcRn mRNA levels for overall survival (OS) of NSCLC patients through the calculation of hazard ratios (HR) at the univariate and multivariate levels. Multivariate models were adjusted for tumor stage, tumor histotype and patient age (model a) or metastasis status, tumor histotype and patient age (model b) (Table 1).

SPSS Statistics (version 17.0), MedCalc software (version 12.5) and GraphPad Prism (version 5.00) were used.

Results:

FcRn mRNA is Down-Regulated in NSCLC Tissue

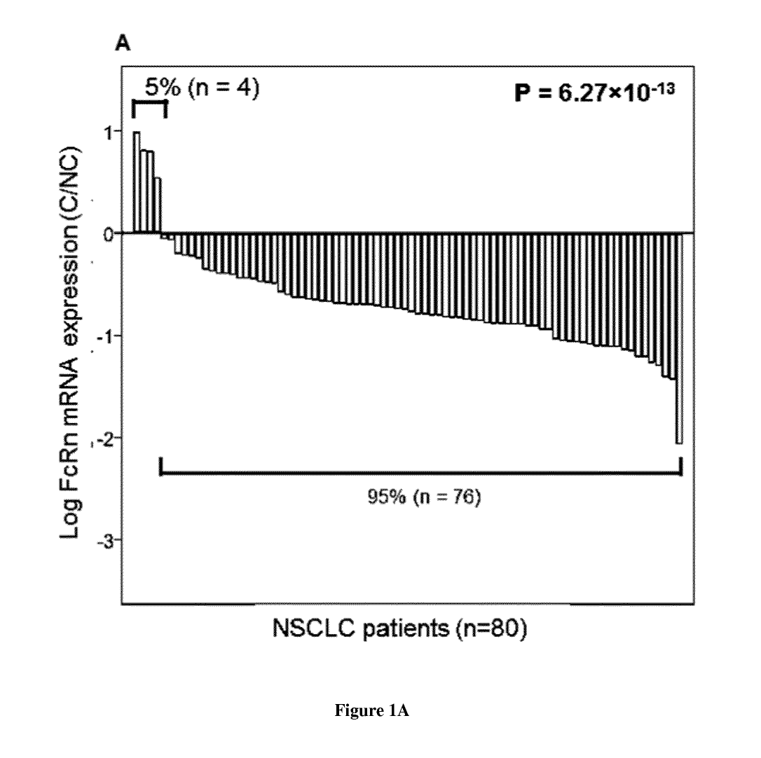

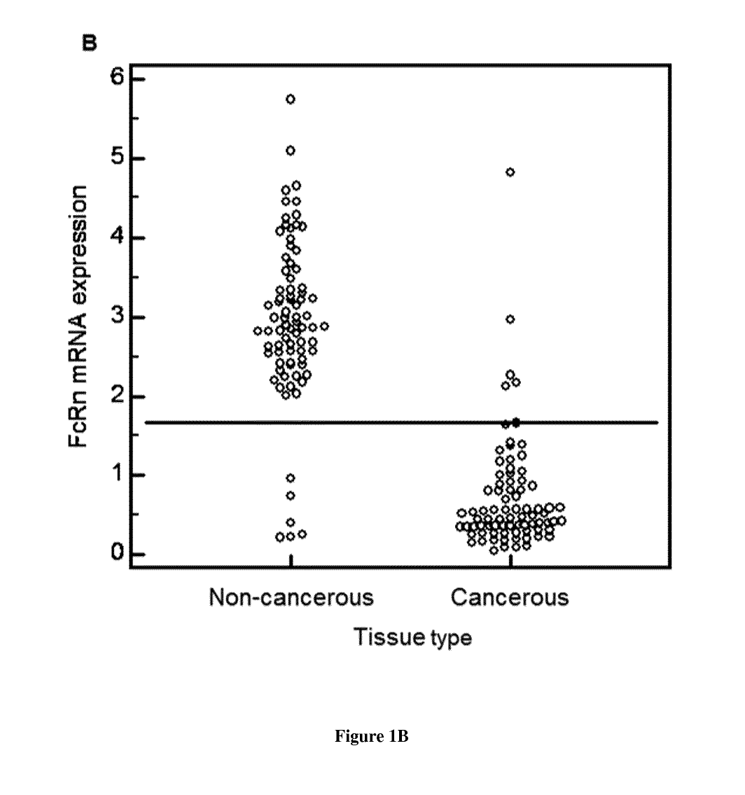

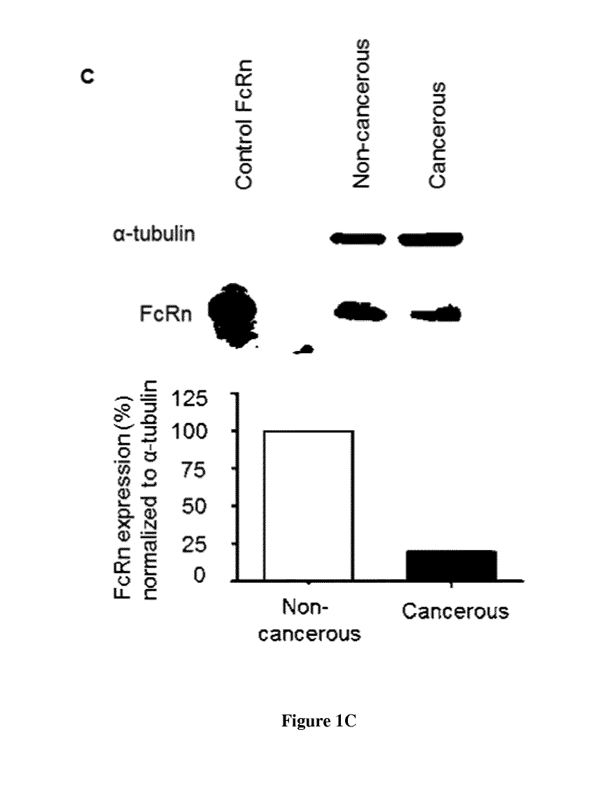

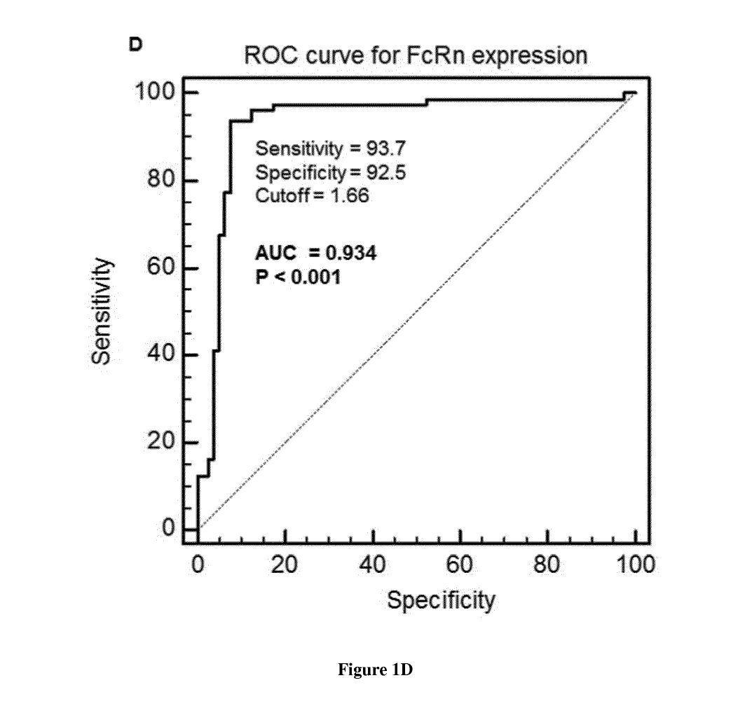

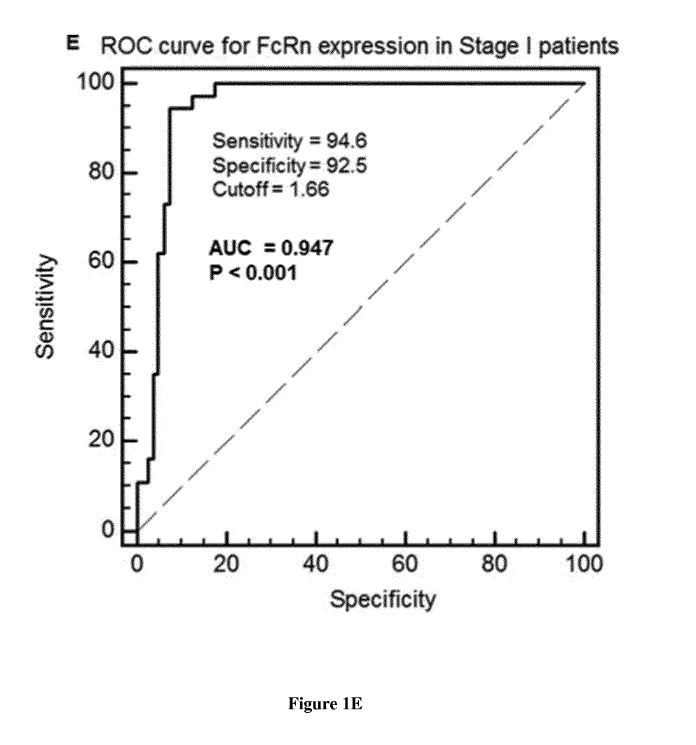

FcRn mRNA was assayed by qRT-PCR in cancerous and non-cancerous samples from patients with NSCLC (FIG. 1A). Mean FcRn mRNA levels were significantly lower in the cancerous (mean±SE=0.727±0.080) than non-cancerous (mean±SE=2.95±0.12) tissue; FcRn mRNA levels were lower in 95% (76/80) of the NSCLC than paired non-cancerous tissues (P<0.001); the median FcRn mRNA values were 6-fold lower in the cancerous than non-cancerous samples (median tumor=0.482, median adjacent normal tissue=2.89; P<0.001) (FIG. 1B). Similarly, western blotting of pooled protein extracts from different patients, chosen randomly, showed that the amount of FcRn (normalized to α-tubulin) was much lower in cancerous than non-cancerous tissue (FIG. 1C). Using a cut-off value of 1.66 expression units, the relative FcRn mRNA levels in cancerous and non-cancerous tissues showed a sensitivity of 93.7% and a specificity of 92.5%, (FIG. 1D). ROC analysis of FcRn mRNA levels (FIG. 1D) produced a notable AUC of 0.934 (SE=0.024, 95% CI=0.884-0.967, P<0.001). At fixed sensitivities of 90.0% and 95.0%, specificity values were 92.50% and 87.50%, respectively. At fixed specificities of 80.0% and 90.0%, sensitivity values were 97.50% and 93.75%, respectively. Findings for stage I specimens were similar: the difference in expression was 5-fold (P<0.001), and the corresponding ROC curve analysis generated an AUC of 0.947 (95% CI=0.905-0.989, P<0.001) (FIG. 1E). No significant association was found between FcRn mRNA levels in cancerous tissues and clinico-pathological features. FcRn mRNA was twice as abundant in stage IB than stage IA samples (P=0.043).

Expression of FcRn in NSCLC Patients is Mainly Attributed to Tumor-Infiltrating Cells

The distribution of FcRn protein has been studied in the normal lungs of various species and is restricted to bronchial epithelial cells and alveolar macrophages in humans¹⁹, NSCLC originate mainly from epithelial bronchial cells (and in some cases from epithelial alveolar cells), so we tested for FcRn by immunohistochemistry in cancerous and non-cancerous lung tissues. We detected FcRn in alveolar macrophages and at very low levels in the bronchial epithelium of the non-cancerous tissue (data not shown). In tumor samples, it was mainly in large cells, located in the immune islets, in the stromal and peri-vascular compartments (data not shown): these cells are probably immune cells, such as dendritic cells, as previously described by Baker et al. in human colorectal carcinomas¹⁵.

Prognostic Value of FcRn mRNA for NSCLC Patients

FcRn mRNA in Cancerous Tissues is Associated with a Favorable Prognosis

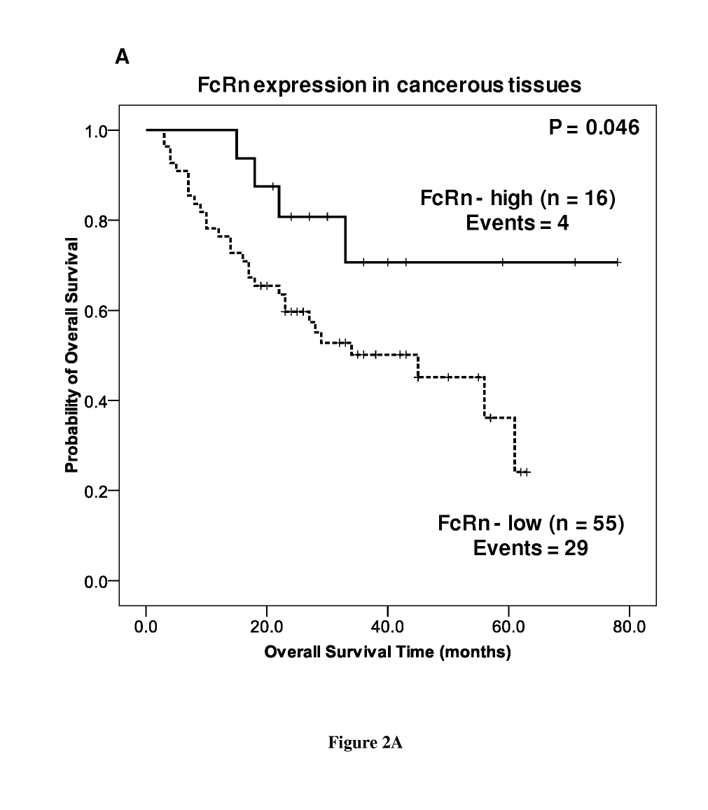

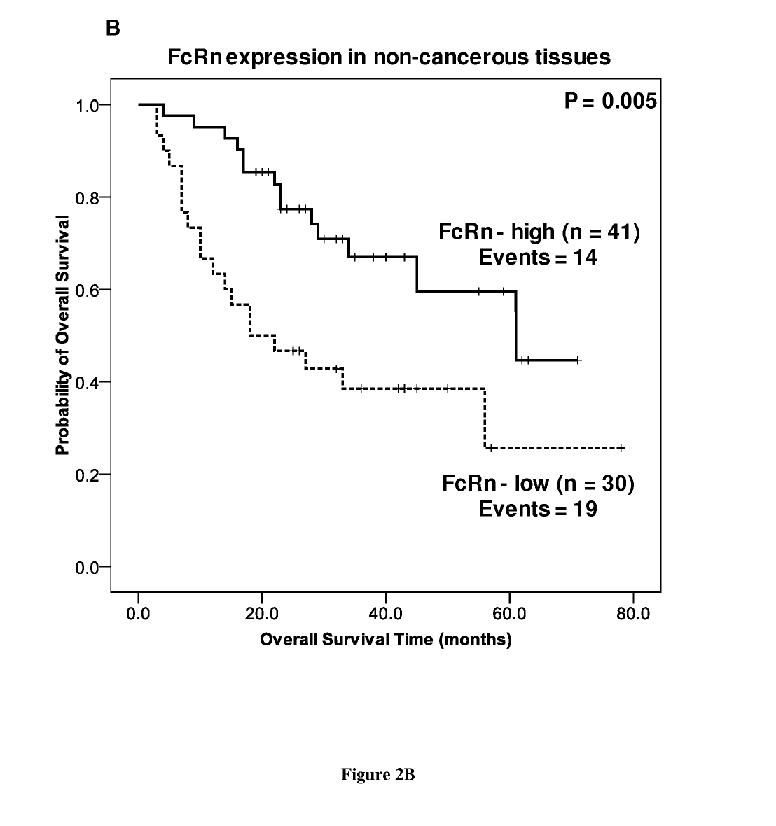

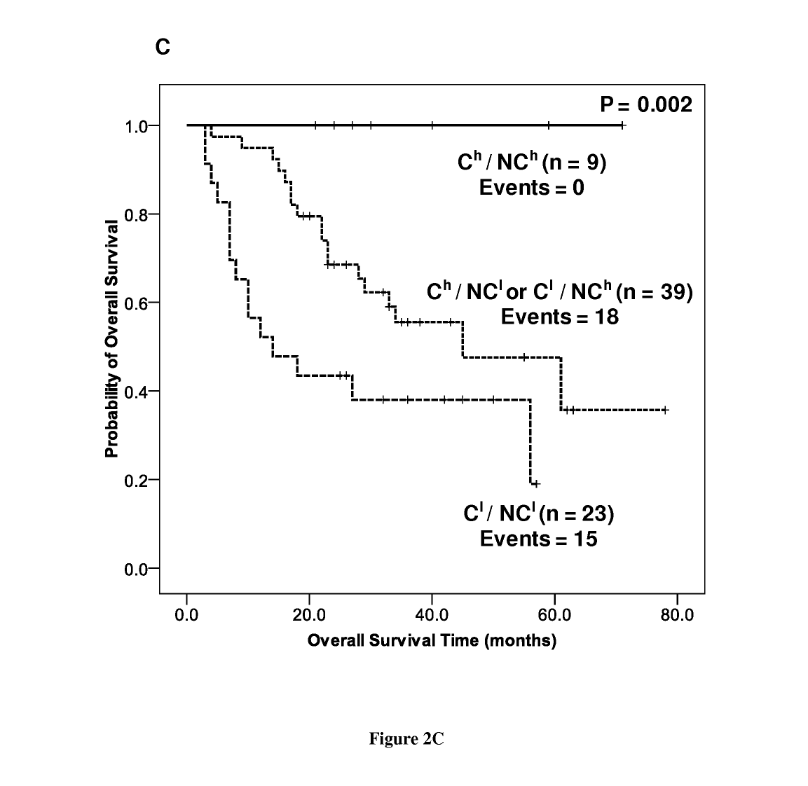

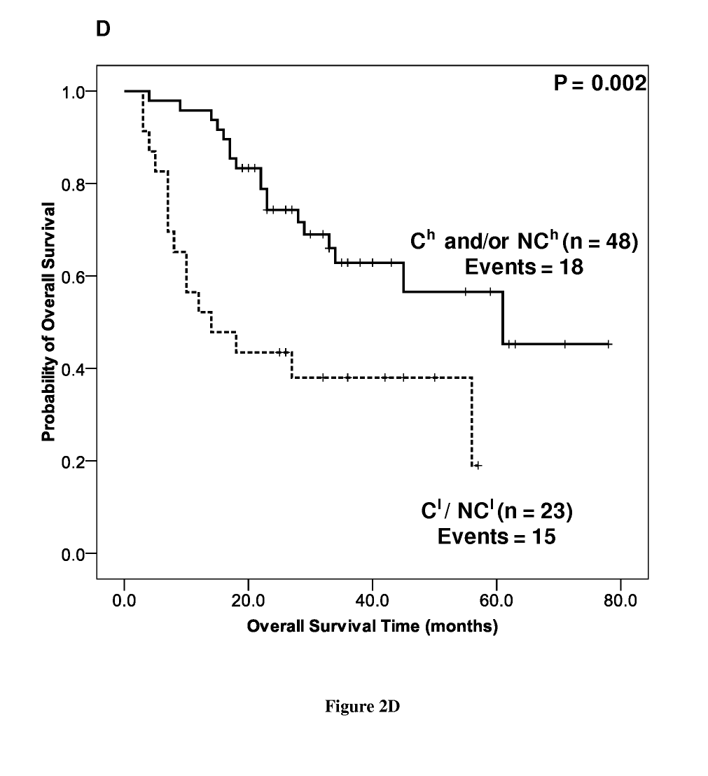

Baker et al. showed that the frequency of specific FcRn-positive cells correlated with survival in colorectal carcinoma, so we evaluated the predictive value of testing for FcRn in cancerous and non-cancerous lung tissues. The predictive value of FcRn for NSCLC patient survival was evaluated by analyzing FcRn mRNA expression (high or low) in cancer tissues. The survival of NSCLC patients classified as FcRn-high (62.0 months (SE=6.9)) was better than that of FcRn-low patients (37.3 months (SE=3.3); P=0.046 Kaplan-Meir analysis) (FIG. 2A). Multivariate Cox regression analysis, adjusted for significant clinicopathological variables, identified FcRn expression in the cancerous tissues as an independent indicator of favorable prognosis for NSCLC patients (HR=0.332, 95% CI=0.112-0.983, P=0.047) (Table 1).

FcRn mRNA in Non-Cancerous Tissues is Also an Independent Predictor of Survival for NSCLC Patients

Taking into consideration the recently described role of FcRn in the anti-tumor immune response¹⁵ and the prognostic gene expression signatures that can be derived from tumor-adjacent tissue parts as previously reported for several human malignancies^{20, 21}, we sought to evaluate the predictive value of FcRn expression in non-cancerous tissues obtained from NSCLC patients. High FcRn mRNA levels in the non-cancerous specimens were associated (P=0.005) with favorable prognosis (FIG. 2B), whereas patients with low FcRn expression were 2.6 times more likely to die of the disease (P=0.007; Table 1). FcRn expression in non-cancerous tissues was found to constitute a strong independent predictor of favorable overall survival outcome in NSCLC patients (HR=0.323, 95% CI=0.154-0.678, P=0.003) (Table 1).

Assessment of FcRn mRNA in Both the Tumor and Non-Cancerous Tissues can Stratify NSCLC Patients According to Overall Survival

We further stratified NSCLC patients, according to FcRn mRNA levels in both cancerous (C) and non-cancerous (NC) tissue. Overall survival periods were longer for high (h) than low (1) FcRn mRNA levels in both cancerous and non-cancerous tissues (P=0.002) (FIG. 2C-D). Strikingly, none of the “double high” (C^{h}/NC^{h}) patients died during the follow-up period, whereas the survival probabilities progressively worsened over time for patients with one or both tissue types scored as FcRn-low (FIG. 2C). The findings were similar when the patients were grouped into those with high FcRn expression in at least one tissue type (C^{h} and/or NC^{h}) and those with low FcRn expression in both tissue types (C^{l}/NC): the latter group showed significantly worse outcomes (P=0.002) (FIG. 2D). Thus, analysis of FcRn expression in both tissues types can provide significant and robust prognostic information (HR=0.273, 95% CI=0.129-0.577, P=0.001 Table 1) about NSCLC patients, which is independent of the currently used conventional indicators and important clinicopathological variables.

Prognostic Value of FcRn mRNA in Early Stage and in Metastasis-Free NSCLC Patients

We analyzed the prognostic performance of FcRn mRNA levels in subgroups of NSCLC patients conventionally classified as “lower-risk”. Kaplan-Meier survival analysis within the early stage (I/II) patient subgroup indicated that high FcRn mRNA levels retained their association with favorable outcome; this was statistically significant for non-cancerous tissue (P=0.035). Early stage patients with high levels of FcRn mRNA in at least one tissue (C^{h} and/or NC^{h}) survived longer than patients with low levels of FcRn mRNA in both tissues (C^{l}/NC^{l}) (P=0.055). Similar observations were made for the non-metastatic patients, as FcRn mRNA expression again endowed NSCLC patients with enhanced overall survival intervals; this held true for the determination of FcRn mRNA expression in non-cancerous tissue parts (P=0.007) and the comparisons between NSCLC patients with at least one tissue part with a high expression versus patients with both tissue having a low expression of FcRn mRNA (P=0.008).

Multivariate analysis confirmed the independent prognostic information from FcRn expression levels in normal and cancerous tissues, further demonstrating the similar yet discrete clinical significance of these assessments.

Prognostic Value of FcRn mRNA Expression is Validated in Others Cohorts

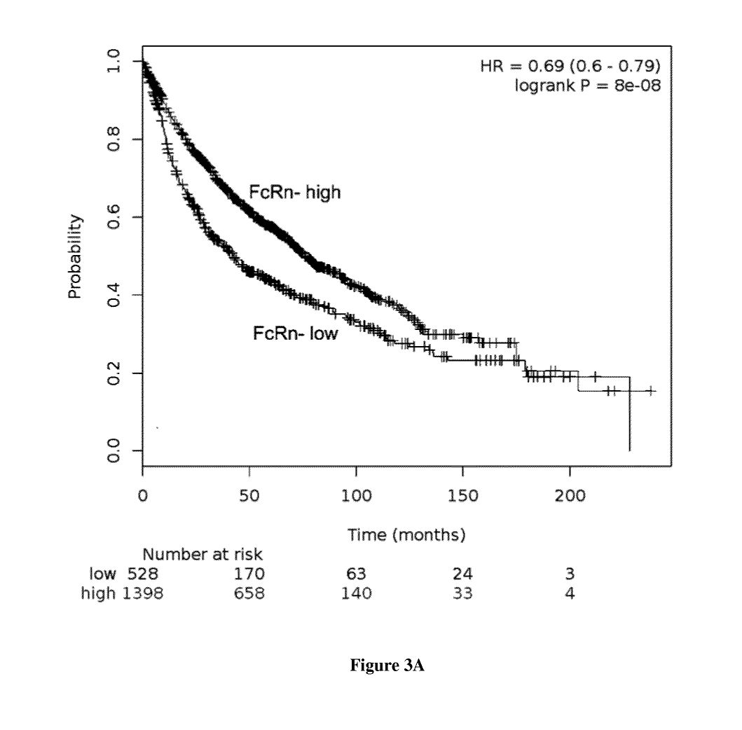

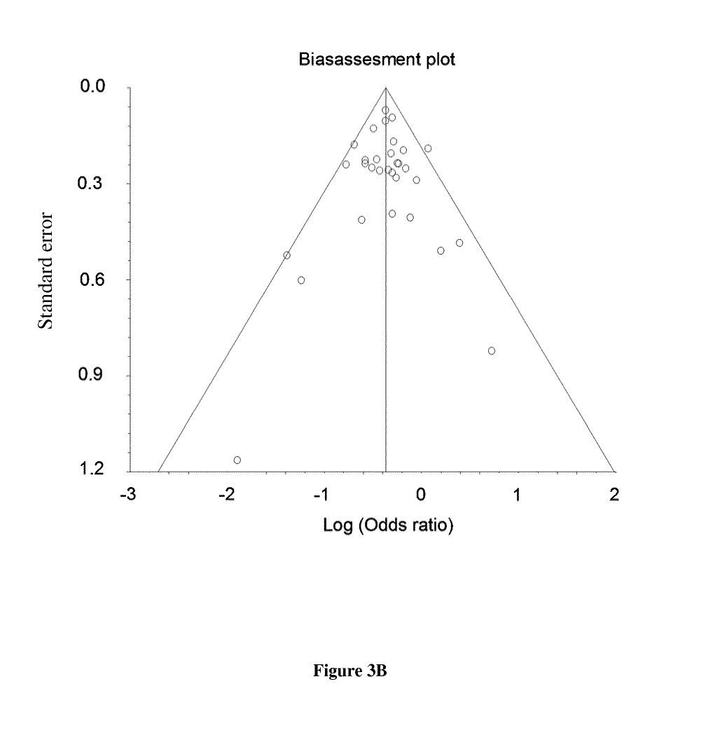

In order to reinforce our findings regarding the prognostic significance of FcRn in lung cancer, we performed in silico analysis of FcRn expression. Firstly, we analyzed microarrays studies extracted from the Oncomine database (23). The results showed that FcRn down regulation was also associated with poor survival of NSCLC patients as found in our qRT-PCR study. Secondly, we used Affymetrix microarray expression data from lung cancer patients and analyzed FcRn expression as determined by probe set 218831_s, based on the online “Kaplan Meier Plotter” tool (24). The significant association of FcRn expression with favorable overall survival of lung cancer patients that we observed, was validated using this independent dataset of 1,926 lung cancer samples (FIG. 3A) at the univariate (HR=0.69, 95% CI=0.6-0.79, P=8×10-8), and multivariate levels after adjustment for stage and tumor histotype (HR=0.69, 95% CI=0.69, P=0.0009). Finally to validate these results, we performed a meta-analysis on data from publicly available expression analysis platforms: Kaplan Meier Plotter (24), PrognoScan (25), PROGgeneV2 (26) and SurvExpress (27). FcRn expression was strongly associated with favorable overall survival of lung cancer patients as indicated by the pooled HR from n=31 studies/databases HR=0.70 (95% CI=0.65-0.74), P<0.0001. In 28/31 studies the HRs were <1 (indicating association with favorable prognosis) and 12/31 were individually statistically significant, including large cohorts from the Kaplan Meier plotter database (N=1926), the SurvExpress platform (N=1044) and the CAARAY, NCI (N=468) (data not shown). Moreover, no statistical heterogeneity was observed (Cochran Q=33.08, df=30, P=0.3191, I²=9.3%, 95% CI=0-42%), neither statistically significant bias (Begg-Mazumdar: Kendall's tau=0.0409 P=0.7616, Egger: bias=−0.04965, 95% CI=−0.790-0.691, P=0.8918) (FIG. 3B). The absence of statistically significant heterogeneity and bias are indicators of the sufficient quality of the meta-analyses and the validity of the deriving conclusions.

### Discussion

The lungs are one of the major organs expressing FcRn and a common site of carcinogenesis. In this study, we showed a significant decrease of FcRn expression, at both the mRNA and protein levels, in the lung cancerous compared to the lung non-cancerous tissues from NSCLC patients. This discriminative value of FcRn between cancer lesions from non-cancerous tissue parts is high, even for early stage patients (AUC=0.947). This result is in line with the recent evidences demonstrating a central role of FcRn in anti-tumor immune-surveillance¹⁵. Herein, we also found that FcRn mRNA levels were associated with a favorable outcome and provided prognostic information independently from important clinico-pathological parameters, when it was assessed in the cancer or, intriguingly, in the non-cancerous tissue part. In fact, the prognostic information derived from the non-cancerous part was even more robust (HR=0.323, 95% CI=0.154-0.678, P=0.003 Table 1). Those results add to the existing evidences on the central role of the tumor's niche on carcinogenesis and disease progression²³.

In colorectal carcinoma, Baker et al. also found that FcRn-positive dendritic cells (DCs) were encountered not only in the cancerous tissue part but also in the adjacent normal stroma. FcRn-positive DCs were also strongly correlated with the presence of CD8+ T cells in the non-cancerous tissue¹⁵. When analyzing the combined non-cancerous and cancerous expression of FcRn, we noticed a progressive deterioration of overall survival periods with the decrease presence of FcRn in non-cancerous and cancerous tissue parts. None of the patients classified as FcRn-high for both tissue parts died within the study's follow-up period, whereas the survival probabilities worsened when moving to patients with one tissue part and both tissue parts showing low expression (P=0.002). This observation extends the tumor-protective role of FcRn in the lungs.

The combined analysis of FcRn expression can provide significant and robust prognostic information (HR=0.273, 95% CI=0.129-0.577, P=0.001 Table 1) regarding the overall survival of NSCLC patients. The robustness of our findings is clearly reinforced by the fact that same results were found in other independent and international cohorts. It was found to be independent of the currently used conventional indicators and important clinico-pathological parameters. Furthermore, FcRn expression, especially when assessed in the non-cancerous tissue part has prognostic relevance even for the “lower-risk” early stage (P=0.035) and metastasis-free NSCLC patients (P=0.007). Interestingly, multivariate analysis also proved the distinctly independent prognostic information deriving from non-cancerous and cancerous FcRn expression, corroborating the similar yet discrete clinical significance of these assessments.

The tumor, node and metastasis (TNM) classification is not enough informative about prognosis and the potential benefits of adapted therapy in NSCLC²⁴. We report the first demonstration of the prognostic and predictive values of testing for FcRn mRNA in NSCLC patients. The prognostic value of testing for FcRn in specific cell populations by double IHC staining has already been described in colorectal carcinoma¹⁵. Here, we showed that the global FcRn mRNA level is a robust and independent marker of NSCLC patient prognosis. Testing for this mRNA is straightforward, and can readily be applied in the clinic. This novel marker could be combined with the recently described Immunoscore®²⁵, a method measuring the beneficial impact of the immune infiltrate on tumour outcome, which has emerged in colorectal cancer and may be relevant in other malignancies, such as lung cancer²⁶. Taking together, they may help with decision-making for NSCLC management, contributing to the timely and appropriate administration of adjuvant treatment.

TABLE 1
Cox regression overall survival analyses at the univariate and multivariate levels

| | Univariate Analysis | | | Multivariate Analysis | | | |
|---|---|---|---|---|---|---|---|
| Variable | HR | 95% CI | P value | Variable | HR | 95% CI | P value |
| FcRn | | | | | | | |
| mRNA level | | | | FcRn | | | |
| in | | | | mRNA level | | | |
| cancerous | | | | in cancerous | | | |
| tissue | | | | tissue | | | |
| Low (l) | 1.00 | | | Low | 1.00 | | |
| High (h) | 0.362 | 0.127-1.03 | 0.057 | High | 0.332^{a} | 0.112-0.983 | 0.047 |
| | | | | | 0.267^{b} | 0.089-0.806 | 0.019 |
| | | | | FcRn | | | |
| FcRn mRNA | | | | mRNA level | | | |
| level in non- | | | | in non- | | | |
| cancerous | | | | cancerous | | | |
| tissue | | | | tissue | | | |
| Low | 1.00 | | | Low | 1.00 | | |
| High | 0.384 | 0.192-0.769 | 0.007 | High | 0.323^{a} | 0.154-0.678 | 0.003 |
| | | | | | 0.339^{b} | 0.162-0.709 | 0.004 |
| FcRnmRN | | | | FcRnmRNA | | | |
| A levels in | | | | levels in | | | |
| both tissue | | | | both tissue | | | |
| types | | | | types | | | |
| C^{l}/NC^{l} | 1.00 | | | C^{l}/NC^{l} | 1.00 | | |
| C^{h}and/or | 0.355 | 0.177-0.714 | 0.004 | C^{h}and/or | 0.273^{a} | 0.129-0.577 | 0.001 |
| NC^{h} | | | | NC^{h} | 0.263^{b} | 0.124-0.556 | <0.001 |
| Stage | 1.22 | 1.04-1.44 | 0.015 | | | | |
| (continuous) | | | | | | | |
| Histotype | | | | | | | |
| SCC | 1.00 | | | | | | |
| ADC | 1.23 | 0.605-2.50 | 0.566 | | | | |
| Age | 0.992 | 0.961-1.02 | 0.596 | | | | |
| (continuous) | | | | | | | |
| Metastasis | | | | | | | |
| No | 1.00 | | | | | | |
| Yes | 2.37 | 1.09-5.17 | 0.030 | | | | |

^{a}Multivariate model adjusted for stage, histotype, age (model a).
^{b}Multivariate model adjusted for metastasis status, histotype, age (model b).

### EXAMPLE 2

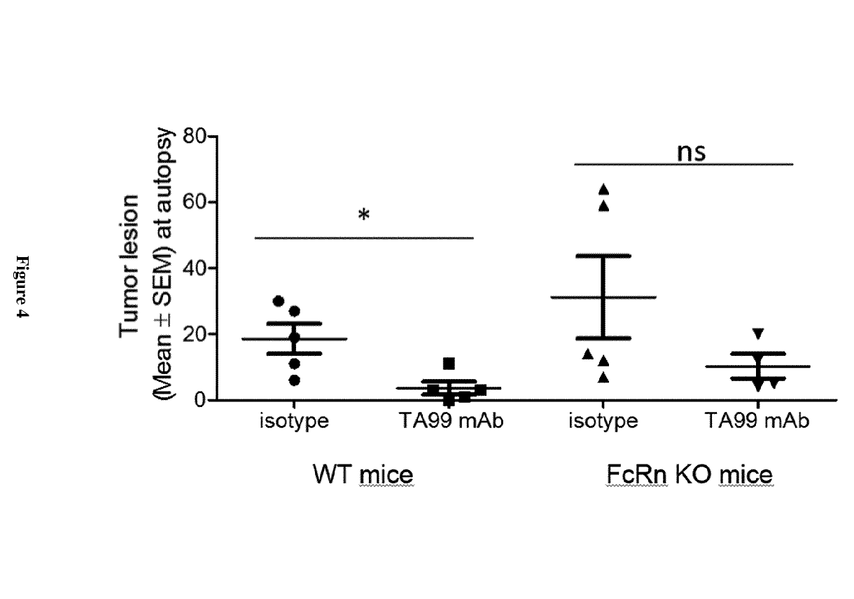

FIG. 4 shows tumor lesions in the lung of wild-type (WT) and FcRN KO mice that were treated with a specific (TA99 mAb) monoclonal antibody and an irrelevant control isotype. B16F10-Luc melanoma cells (100 000 cells/animal) were injected in the tail vein of C57 BL/6 mice. At day 1, day 4 and day 8 after tumor induction animals were treated by i.v. injection with an anti-tyrosinase-related protein-1 monoclonal antibody (200 μg, TA99 from BioXcell) or an irrelevant control isotype (200 μg, IgG2a). Animals were sacrificed at day 18, lung excised and tumor lesions counted. Results are expressed as Mean±SEM and analyzed using a Mann Withney non parametric test.

The results show that treatment of animals that do not express FcRn did not respond to the mAb treatment compared to WT animals.

### REFERENCES

Throughout this application, various references describe the state of the art to which this invention pertains. The disclosures of these references are hereby incorporated by reference into the present disclosure.
1. Raghavan M, Gastinel L N, Bjorkman P J: The class I major histocompatibility complex related Fc receptor shows pH-dependent stability differences correlating with immunoglobulin binding and release. Biochemistry (Mosc) 32:8654-8660, 1993
2. Roopenian D C, Akilesh S: FcRn: the neonatal Fc receptor comes of age. Nat Rev Immunol 7:715-725, 2007
3. Ward E S, Ober R J: Chapter 4: Multitasking by exploitation of intracellular transport functions the many faces of FcRn. Adv Immunol 103:77-115, 2009
4. Claypool S M, Dickinson B L, Wagner J S, et al: Bidirectional transepithelial IgG transport by a strongly polarized basolateral membrane Fcgamma-receptor. Mol Biol Cell 15:1746-1759, 2004
5. Junghans R P, Anderson C L: The protection receptor for IgG catabolism is the beta2-microglobulin-containing neonatal intestinal transport receptor. Proc Natl Acad Sci USA 93:5512-5516, 1996
6. Roopenian D C, Christianson G J, Sproule T J, et al: The MHC class I-like IgG receptor controls perinatal IgG transport, IgG homeostasis, and fate of IgG-Fc-coupled drugs. J Immunol Baltim Md 1950 170:3528-3533, 2003
7. Ben Suleiman Y, Yoshida M, Nishiumi S, et al: Neonatal Fc receptor for IgG (FcRn) expressed in the gastric epithelium regulates bacterial infection in mice. Mucosal Immunol 5:87-98, 2012
8. Yoshida M, Kobayashi K, Kuo T T, et al: Neonatal Fc receptor for IgG regulates mucosal immune responses to luminal bacteria. J Clin Invest 116:2142-2151, 2006
9. Bai Y, Ye L, Tesar D B, et al: Intracellular neutralization of viral infection in polarized epithelial cells by neonatal Fc receptor (FcRn)-mediated IgG transport. Proc Natl Acad Sci USA 108:18406-18411, 2011
10. Ko S-Y, Pegu A, Rudicell R S, et al: Enhanced neonatal Fc receptor function improves protection against primate SHIV infection. Nature 514:642-645, 2014
11. Zhu X, Meng G, Dickinson B L, et al: MHC class I-related neonatal Fc receptor for IgG is functionally expressed in monocytes, intestinal macrophages, and dendritic cells. J Immunol Baltim Md 1950 166:3266-3276, 2001
12. Vidarsson G, Stemerding A M, Stapleton N M, et al: FcRn: an IgG receptor on phagocytes with a novel role in phagocytosis. Blood 108:3573-3579, 2006
13. Qiao S-W, Kobayashi K, Johansen F-E, et al: Dependence of antibody-mediated presentation of antigen on FcRn. Proc Natl Acad Sci USA 105:9337-9342, 2008
14. Baker K, Qiao S-W, Kuo T T, et al: Neonatal Fc receptor for IgG (FcRn) regulates cross-presentation of IgG immune complexes by CD8-CD11b+ dendritic cells. Proc Natl Acad Sci USA 108:9927-9932, 2011
15. Baker K, Rath T, Flak M B, et al: Neonatal Fc receptor expression in dendritic cells mediates protective immunity against colorectal cancer. Immunity 39:1095-1107, 2013
16. Sörenson S, Glimelius B, Nygren P, et al: A systematic overview of chemotherapy effects in non-small cell lung cancer. Acta Oncol Stockh Swed 40:327-339, 2001
17. Gueugnon F, Barascu A, Mavridis K, et al: Kallikrein-related peptidase 13: an independent indicator of favorable prognosis for patients with nonsmall cell lung cancer. Tumour Biol J Int Soc Oncodevelopmental Biol Med, 2015
18. DeLong E R, DeLong D M, Clarke-Pearson D L: Comparing the areas under two or more correlated receiver operating characteristic curves: a nonparametric approach.

Biometrics 44:837-845, 1988
19. Spiekermann G M, Finn P W, Ward E S, et al: Receptor-mediated immunoglobulin G transport across mucosal barriers in adult life: functional expression of FcRn in the mammalian lung. J Exp Med 196:303-310, 2002
20. Romin-Perez E, Casbas-Hernindez P, Pirone J R, et al: Gene expression in extratumoral microenvironment predicts clinical outcome in breast cancer patients. Breast Cancer Res BCR 14:R51, 2012
21. Lamb J R, Zhang C, Xie T, et al: Predictive genes in adjacent normal tissue are preferentially altered by sCNV during tumorigenesis in liver cancer and may rate limiting. PloS One 6:e20090, 2011
22. Györffy B, Surowiak P, Budczies J, et al: Online survival analysis software to assess the prognostic value of biomarkers using transcriptomic data in non-small-cell lung cancer. PloS One 8:e82241, 2013
23. Heinrich E L, Walser T C, Krysan K, et al: The inflammatory tumor microenvironment, epithelial mesenchymal transition and lung carcinogenesis. Cancer Microenviron Off J Int Cancer Microenviron Soc 5:5-18, 2012
24. Wang J, Wu N, Zheng Q, et al: Evaluation of the 7th edition of the TNM classification for lung cancer at a single institution. J Cancer Res Clin Oncol 140:1189-1195, 2014
25. Anitei M-G, Zeitoun G, Mlecnik B, et al: Prognostic and predictive values of the immunoscore in patients with rectal cancer. Clin Cancer Res Off J Am Assoc Cancer Res 20:1891-1899, 2014
26. Galon J, Mlecnik B, Bindea G, et al: Towards the introduction of the “Immunoscore” in the classification of malignant tumours. J Pathol 232:199-209, 2014
27. Paintaud G. Pharmacokinetics (PK) of mAbs Med Sci (Paris). 2009 December; 25(12):1057-62.
28. Keizer R J, Huitema A D, Schellens J H, Beijnen J H. Clinical pharmacokinetics of therapeutic monoclonal antibodies. Clin Pharmacokinet. 2010 August; 49(8):493-507.
29. Datta-Mannan A, Wroblewski V J. Application of FcRn binding assays to guide mAb development. Drug Metab Dispos. 2014 November; 42(11):1867-72.
30. Azzopardi N, Lecomte T, Ternant D, Boisdron-Celle M, Piller F, Morel A, Gouilleux-Gruart V, Vignault-Desvignes C, Watier H, Gamelin E, Paintaud G. Cetuximab pharmacokinetics influences progression-free survival of metastatic colorectal cancer patients. Clin Cancer Res. 2011 Oct. 1; 17(19):6329-37
31. Ternant D, Ducourau E, Fuzibet P, Vignault C, Watier H, Lequerre T, Le Loet X, Vittecoq O, Goupille P, Mulleman D, Paintaud G. Pharmacokinetics and concentration-effect relationship of adalimumab in rheumatoid arthritis. Br J Clin Pharmacol. 2015 February; 79(2):286-97.
32. Rath T, Baker K, Pyzik M, Blumberg R S. Regulation of immune responses by the neonatal fc receptor and its therapeutic implications. Front Immunol. 2015 Jan. 5; 5:664.
33. Pyzik M, Rath T, Lencer W I, Baker K, Blumberg R S. FcRn: The Architect Behind the Immune and Nonimmune Functions of IgG and Albumin. J Immunol. 2015 May 15; 194(10):4595-603.
34. Ye L, Zeng R, Bai Y, Roopenian D C, Zhu X. Efficient mucosal vaccination mediated by the neonatal Fc receptor. Nat Biotechnol. 2011 February; 29(2):158-63
35. Lu L, Palaniyandi S, Zeng R, Bai Y, Liu X, Wang Y, Pauza C D, Roopenian D C, Zhu X A neonatal Fc receptor-targeted mucosal vaccine strategy effectively induces HIV-1 antigen-specific immunity to genital infection. J Virol. 2011 October; 85(20):10542-53.

1. A method for predicting the survival time of a patient suffering from a solid cancer comprising i) determining in a tissue sample obtained from the patient the gene expression level of neonatal Fc receptor (FcRn) ii) comparing the expression level determined at step i) with its predetermined reference value and iii)
providing a good prognosis when the expression level determined at step i) is higher than the predetermined reference value, or
providing a bad prognosis when the expression level determined at step i) are is lower than the predetermined reference value.

2. The method according to claim 1, wherein the tissue sample is a non-tumor sample or a tumor sample.

3. The method according to claim 2, wherein the tissue sample is non-tumor sample.

4. A method for predicting the survival time of a patient suffering from a solid cancer comprising i) determining in a non-tumor sample and in a tumor sample obtained from the patient the gene expression level of neonatal Fc receptor (FcRn) ii) comparing every expression level determined at step i) with its predetermined reference value and iii)
providing a good prognosis when both expression levels determined at step i) in a non-tumor sample and in a tumor sample are higher than their predetermined reference values, or
providing a bad prognosis when both expression levels determined at step i) in a non-tumor sample and in a tumor sample are lower than their predetermined reference values or
providing an intermediate prognosis when at least one expression level determined at step i) in a non-tumor sample or in a tumor sample is higher than its predetermined value.

5. The method according to claim 1, wherein the solid tumor is lung cancer.

6. The method according to claim 5, wherein the lung cancer is non-small cell lung cancer.

7. A method for determining whether a patient with a solid cancer will respond to a treatment comprising i) determining in a tumor sample obtained from the patient the gene expression level of FcRn ii) comparing the expression level determined at step i) with a predetermined reference value and iii) concluding that the patient will significantly respond to the treatment when the expression level determined at step i) is higher than the predetermined reference value, or concluding that the patient will not significantly respond to the treatment when the expression level determined at step i) is lower than the predetermined reference value.

8. The method according to claim 7, wherein the solid cancer is lung cancer.

9. The method according to claim 8 wherein the lung cancer is non-small cell lung cancer.

10. The method according to claim 7, wherein the treatment is immunotherapeutic treatment.

11. The method according to claim 1 further comprising a step of measuring an expression level of additional biological markers related to the adaptive immune response within the tumour.

12. A kit for predicting the survival time according to claim 1, comprising:
at least a means for determining the expression level of neonatal Fc receptor (FcRn) and
instructions for use.

13. A kit according to claim 12, comprising:
amplification primers and/or at least one probe for determining the expression level of neonatal Fc receptor (FcRn), and
instructions for use.

14. The method of claim 1, further comprising, if the patient has a good prognosis, providing the patient with one or more of radiotherapy, chemotherapy and immunotherapy.

15. The method of claim 7, further comprising, if it is determined that the patient will significantly respond to the treatment, providing the patient with the treatment.

16. The method of claim 15, wherein the treatment is one or more of radiotherapy, chemotherapy and immunotherapy.


SEQUENCE LISTING (2 sequences)

SEQ ID NO: 1 - DNA, 19 bp, Artificial
  Synthetic Forward primer
ccctggcttttccgtgctt

SEQ ID NO: 2 - DNA, 19 bp, Artificial
  Synthetic Reverse primer
tgacgattcccaccacgag